(19) United States
(12) Patent Application Publication
(10) Pub. No.: US 2021/0068711 A1
(43) Pub. Date: Mar. 11, 2021

(54) COMPUTERIZED REWARD SYSTEM OF DIGITAL ASSETS

(71) Applicant: Aldric TRINDADE NEGRIER, Faro (PT)

(72) Inventors: Aldric TRINDADE NEGRIER, Faro (PT); Luis Carlos RODRIGUES DE SOUSA, Luis (PT); Samuel RAMOS, Almancil (PT)

(21) Appl. No.: 16/770,607

(22) PCT Filed: Dec. 4, 2018
(86) PCT No.: PCT/IB2018/059617

§ 371 Date: Jun. 7, 2020

(30) Foreign Application Priority Data
Dec. 6, 2017 (PT) 110440

(51) Int. Cl. A61B 5/11; G16H 20/30; G16H 50/30; G16H 40/67; A61B 5/113; G06Q 20/06; H04W 4/80

(52) U.S. Cl. CPC A61B 5/1116; G16H 20/30; G16H 50/30; H04W 4/80; A61B 5/1112; A61B 5/113; G06Q 20/0655; G16H 40/67

(57) ABSTRACT

The present application describes a computerized reward system based on decentralized ledger block-chain technology for rewarding user behaviour beneficial to health. The system is comprised by a monitoring unit (2) that includes sensor means adapted to collect physical parameters (6) from a user (1), in order to create a physical activity record (3), a validation unit (4) with processor means programmed to correlate the parameters of the physical activity record (3) in order to detect the execution of a physical activity by the user (1) and a reward dispensing system (5) comprising a peer-to-peer network configured to manage a decentralized ledger blockchain distributed computing platform for inter-node communication. Said reward dispensing system (5) is programmed to generate and distribute peer-to-peer digital asset rewards (8) in case of detection of a valid physical activity (7) executed by the user (1).

### TECHNICAL FIELD

The present application generally relates to remote monitoring systems, and in particular to a computerized reward system of digital assets for rewarding user behaviour beneficial to health.

### PRIOR ART

Global human population count today, is around 7.6 Billion. Most projections estimate a rise to more than 9.8 Billion before the year 2050 and 11.2 billion by the year 2100 [1]. Moving forward in time, the rapid rate of innovation and technology in fields such as robotics, automation and artificial intelligence will most likely increase the rate of human technological unemployment [2].

In the past 50 years alone we humans have consumed more resources than the whole of humanity before us [3]. This ongoing direction will only increase the strain on public health as well as on the planets eco systems.

The fundamental problem surrounding global warming and all its consequences is not just CO2 emissions alone, it is the logic of endless growth that is built into our problematic economic systems [4]. This endless growth target is un-sustainable in a finite resource planet and has overall very negative effects on our social and ecological systems.

Regarding Public health, several scientific studies now show that stress related problems account for most of hospital visits [5], making it a very serious Public Health concern. Several natural solutions are available to help reduce stress and anxiety, and today, it is a well-known scientific fact that regular breathing, meditation and Yoga practice reduces the body's stress response, by strengthening the relaxation response and lowering stress hormones like cortisol. For this reason today we see the rise in demand for yoga and meditation classes worldwide [6].

In connection to this, solutions exist in the art, such as the case of patent application US20170177833 A1, which discloses an apparatus for implicit triggering of feedbacks relating to user's physical activities, including detecting scanning, in real-time, of a body of a user during one or more physical activities, such as yoga, gymnastics, weight lifting. The application WO2013113036 A1, discloses a system for processing motion-related sensor data for health application, examining motion-related signal from a physical activity sensor located on a subject on a frame-by-frame basis to detect physical activities performed by the subject. The motion-sensing and motion-recognition algorithm of the sensor physical activity data processing engine is able to recognize a basis set of motions into which all human motions can be categorized due to skeleton-connective tissue structures of human anatomy. None of the existing solutions complements the physical activity monitoring with a reward mechanism to motivate users in practicing regular breathing, meditation, yoga practices or other physical activity that can be monitored.

In the context of reward systems, the application U.S. Pat. No. 6,151,586 A provides a computerized reward system for encouraging an individual to participate in a customized health management program, comprising monitoring means for collecting compliance data on an individual participating in the health management program, memory means for storing the compliance data, evaluation means for comparing the compliance data with evaluation criteria to determine whether or not the individual is compliant, and a reward to be given to the individual who is deemed compliant. The reward given to the compliant individual can be a coupon, an electronic reward credited to a data card, or an electronic reward credited to the individual's account at a participating financial institution or retail account. However, none of the reward systems known from the art is based on digital assets and implemented by means of a decentralized server to perform rewards distribution.

### SUMMARY

It is therefore an object of the present application to describe a computerized reward system based on decentralized ledger blockchain technology for rewarding user behaviour beneficial to health, such as long-term breathing, meditation or yoga practices, all activities with scientifically proven health benefits.

To accomplish that, the computerized reward system distributes digital assets to users that have performed physical activities validated by the system. The system is comprised by a monitoring unit that includes sensor means adapted to collect physical parameters from a user, such as physiological, motion and body posture parameters. A validation unit with processor means programmed to correlate the parameters collected by the monitoring unit, is used to detect the execution of a physical activity by the user. Such detection is performed comparing the correlated parameters with physical activity performance models used to characterize all of the physical activities admitted by the system.

To implement an user's reward scheme, the system further comprises a reward dispensing system comprising a peer-to-peer network configured to manage a decentralized ledger blockchain distributed computing platform for inter-node communication. Said reward dispensing system is programmed to generate and distribute peer-to-peer digital asset rewards in case of detection of a physical activity executed by the user.

In an advantageous configuration of the computerized reward system now disclosed, the monitoring unit comprises a body posture sensorial module and a respiratory effort sensor. The body posture sensorial module is adapted to collect inertial measurements, being comprised by a set of accelerometers, gyroscopes and magnetometers. The respiratory effort sensor is configured to detect the inhaling and exhaling breathing periods, and together with the body posture sensorial module data, allow the monitoring unit to collect a complete set of parameters, used to characterized the execution of a certain physical activity, that will be used by the validation unit. In another advantageous mode of the computerized reward system now disclosed, the monitoring unit is enclosed in a wearable strap preferably to be attached to the chest or stomach of a user.

The present application also describes a method for operating the computerized reward system described, said method being comprised the following steps:
Monitoring unit collects physical parameters from a specific user, generating a physical activity record;
Validation unit performs a physical activity validation by matching standard key-parameters of a special correlation scheme with key-parameters resulting from said special correlation scheme applied to the physical activity record;
If the physical activity record is valid, meaning that for a special correlation scheme, a match was detected between the respective standard key-parameters and the key-parameters resulting from correlation of the physical activity record, the physical activity record assigned to the specific user is uploaded to a reward dispensing system;
Reward dispensing system generates and distributes digital asset rewards to the specific user.
   In an advantageous configuration of the method, the physical activity is performed by the user during a physical activity session, said session comprising an initial stage and a final stage during which the monitoring unit collects physical parameters to generate a physical activity session record.

### DESCRIPTION OF FIGURES

FIG. 1 illustrates a block diagram of the computerized reward system of digital assets, wherein the reference signs represents:

1—user;

2—monitoring unit;

3—physical activity record;

4—validation unit;

5—reward dispensing system;

6—physical parameters of the user;

7—valid physical activity record;

8—digital asset reward.

FIG. 2 shows a flow chart representing the operation of the computerized reward system of digital assets, of the present application.

FIG. 3 illustrates an embodiment of the monitoring unit of the computerized reward system, wherein the reference signs represent:

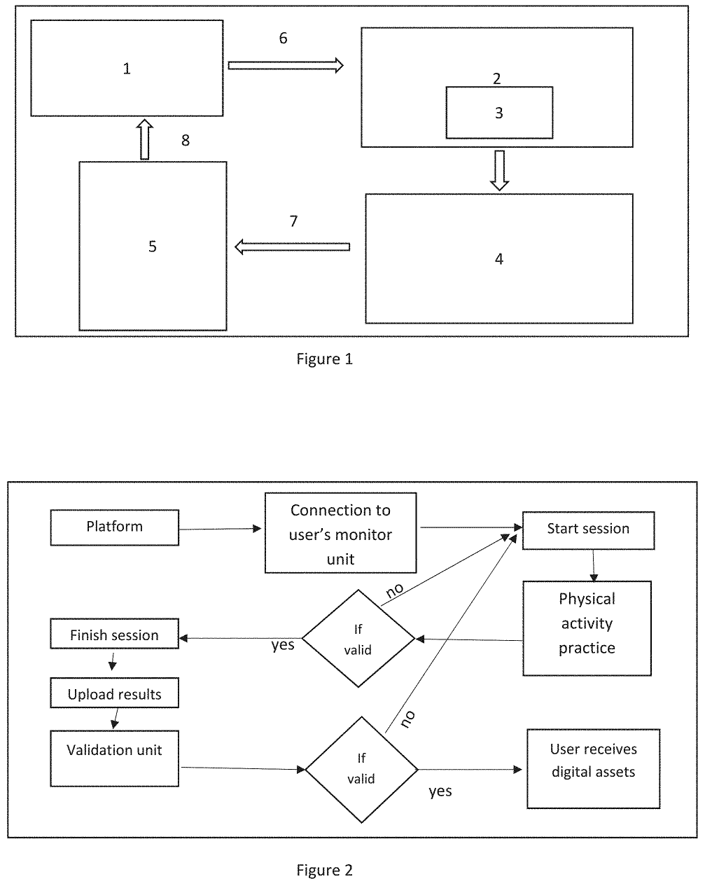

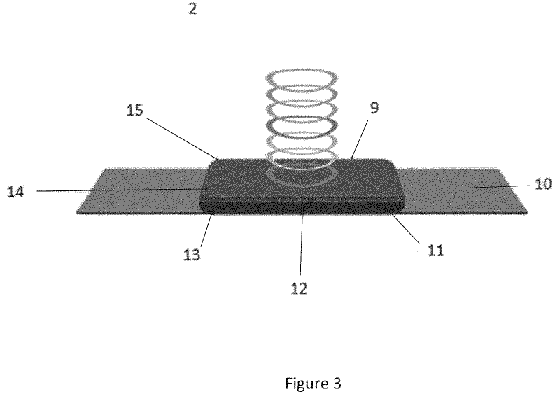

2—monitoring unit;

9—interactive LED;

10—strap;

11—respiratory effort sensor;

12—battery;

13—inertial measurement unit;

14—communication module;

15—vibration means.

### DETAILED DESCRIPTION

The more general and advantageous configurations of the technology now developed are described in the Summary. Such configurations are detailed below in accordance with other advantageous and/or preferred embodiments of implementation of the technology developed.

In a preferred embodiment, the computerized reward system of digital assets of the present application uses a decentralized ledger blockchain technology in order to generate and distribute peer-to-peer digital assets, rewarding user behaviour beneficial to health.

The system comprises a monitoring unit including sensor means adapted to collect physical parameters from a user. Said physical parameters can be related to physiological, motion and body posture, and whose correlation allows to characterize and identify physical activities. In the context of the present application, a physical activity is any bodily movement produced by the skeletal muscles that requires energy expenditure, and encompasses all the activities, at any intensity, able to be monitored, such as but not limited to gardening, farming, cooking, cleaning, long-term breathing, meditation or yoga practices. In order to collect the desired set of parameters, the monitoring unit can be comprised by a body posture sensorial module and a respiratory effort sensor. This set of sensors are capable of detecting user's posture using an inertial measurement unit, comprised by at least one accelerometer, at least one gyroscope and at least one magnetometer, and the expansion and contraction of the chest/stomach. The monitoring unit may also comprise a communication module programmed to establish a bidirectional wireless communication link with an external device, such as a smartphone or any other type of processing device adapted to control the operation of the monitoring unit through said bidirectional wireless communication link. The communication link can be performed over a wide area network, a local area network or a personal area network, such as Bluetooth. In one embodiment of the monitoring unit, the sensor means adapted to collect physical parameters are enclosed in a wearable strap attachable to the chest or stomach of the user. In this embodiment, the monitoring unit further comprises battery means, vibration means and an interactive LED installed in its surface. Yet in another embodiment of the monitoring unit, it is further comprised by a meditation pad, suitable to perform meditation practices, being a smart interactive meditation pad, equipped with LEDs for interactive chromo therapy sessions. Said meditation pad includes a proximity sensor that detects if the user is sitting on it, battery means, vibration means and an illuminated surface configured to emit visible light for relaxing purposes, and wherein the visible light emitted by the pad's surface can vary according to the parameters collected from the respiratory effort sensor. The interactive meditation Pad is equipped with all visible light spectrum range with 16.8 Million possible colors. These colors are used to create a relaxing and peaceful atmosphere that will help create a peaceful state of mind, necessary for an effective guided meditation session.

The validation unit comprises processor means programmed to correlate the parameters collected by the monitoring unit in order to detect the execution of a physical activity by the user. To achieve this purpose, the processor means is comprised by a database structure and a correlation engine. The database structure is configured to store physical activity performance models characterizing all the physical activities admitted by the system. A physical activity performance model is formed by a set of standard key-parameters that identifies unequivocally a physical activity, and result from a special correlation scheme applied to certain physical parameters of the user. The correlation engine is programmed to correlate the physical parameters collected from the user according to special correlating schemes, in order to access if the resultant key-parameters match with the respective standard key-parameters of a physical activity performance model. The physical parameters correlated can be any of the parameters collected from the respiratory effort sensor, from the set of at least one accelerometer, at least on gyroscope and/or at least one magnetometer.

The reward dispensing system, is in communication with the validation unit, and is comprised by a peer-to-peer network configured to manage a decentralized ledger blockchain distributed computing platform for inter-node communication. The reward dispensing system is programmed to generate and distribute peer-to-peer digital asset rewards if the validation unit detects a physical activity executed by the user. The digital asset can be anything that exists in a binary format and can include but are not exclusive to digital documents, audible content, motion picture and other relevant digital data such as a cryptocurrency. In one embodiment of the system the decentralized ledger blockchain distributed computing platform is Ethereum based and the digital asset is Ether.

The computerized reward system further comprises a web user interface electronic platform, provided in a network-based system. Said platform is suitable to provide user access to reward system contents by means of a web application configured to communicate with said network-based system. The reward system contents can be related to activity session records, physical activity guiding sessions and reward data records. Besides that, the platform is capable of connecting users who want to practice physical activities together, by attending to group sessions. In one embodiment, the dispensing system rewards the users of a group practice in proportion of the size of the group. In order to identify a group practice, the system will validate the users that have performed the respective enrolment through the platform. All the group sessions are registered in the platform along with its respective GPS locations.

The present application also describes a method for operating the computerized reward system described above. The monitoring unit collects physical parameters from a specific user and generates a physical activity record. Then, the validation unit performs a physical activity validation comprised by two stages: first, the parameters of the physical activity record are correlated according to special correlation schemes. Each correlation scheme has associated standard key-parameters defining a physical activity performance model, that is stored in the database structure of the validation unit, which unequivocally identify the execution of a physical activity by the user being monitored. In stage two, said standard key-parameters are matched with the correlation results of stage one, in order to access if the user is performing or not the physical activity characterized by the physical activity performance model. Therefore, a physical activity record is considered valid when, for a special correlation scheme, a match was detected between the respective standard key-parameters and the key-parameters resulting from correlation of the physical activity record. In this case, the physical activity record is uploaded to a reward dispensing system which will generate and distribute digital asset rewards to the specific user.

In another embodiment of the method, a physical activity is performed by the user during a physical activity session, comprising an initial stage and a final stage during which the monitoring unit collects physical parameters to generate a physical activity session record. In this context, the validation unit performs a physical activity session validation, by correlating the parameters of the physical activity session record. The physical activity session is validated if the validation unit, for a special correlation scheme, detects a match between the key-parameters resulting from the correlation of the physical activity session record and the standard key-parameters associated to the respective physical activity performance model. In this case, the physical activity session record assigned to the specific user is uploaded to a reward dispensing system. The initial and the final stage can be defined automatically by the monitoring unit when the parameters being monitored vary around a threshold considered as resting level. In another embodiment, the user interacts directly with the monitoring unit in order to define the initial and the final stage of a session.

In another embodiment the amount of digital asset rewards that a specific user receives is a function between the total number of users with valid physical activity record or valid physical activity session record and the daily number of digital assets that the reward dispensing system generates. The digital asset is distributed to the users in periodic batches, for example with a daily frequency.

As an example of the operation of the computerized reward system, it is considered a user performing a meditation practice. Before each meditation practice, the users starts off with a breathing exercise, so the system validates the breathing exercise. After this phase the data coming from the body posture sensorial module ensures that the user is not moving abruptly. Combining the data from the respiratory effort sensor and the body posture sensorial module, according to a specific correlation scheme, the validation unit is able to detect a meditation practice. In case of a Yoga practice, the body posture sensorial module measures and reports a body's specific force, angular rate, and the magnetic field surrounding the body, using a combination of accelerometers, gyroscopes and also magnetometers. The users starts a guided Yoga session using a smartphone, after the session starts, the user mimics the guided Yoga positions, while the system verifies the data from the inertial measurement unit and the respiratory effort sensor. By combining the system with a smartphone, the user is able to access to interactive contents, such as physical activities guided lessons and to manage his physical activity records progression during time.

As will be clear to one skilled in the art, the present invention should not be limited to the embodiments described herein, and a number of changes are possible which remain within the scope of the present invention.

Of course, the preferred embodiments shown above are combinable, in the different possible forms, being herein avoided the repetition all such combinations.

### REFERENCES

[1] United Nations Department of economic and social affairs, World Population Prospects: The 2017 Revision, https://esa.un.org/unpd/wpp/Publications/Files/WPP2017_KeyFindings.pdf
[2] McKinsey Global Institute, A Future That Works: Automation, Employment, and Productivity, https://tinyurl.com/ybbywquw
[3] Australian Academy of Science, Population and environment: a global challenge, Professor Stephen Dovers. Director, Fenner School of Environment and Society, College of Medicine, Biology & Environment, Australian National University
[4] A Critical Analysis of Chromo therapy and Its Scientific Evolution. Samina T. Yousuf Azeemi* and S. Mohsin Raza
[4] University of Michigan, Global warming: New research blames economic growth http://ns.umich.edu/new/releases/20369-global-warming-new-research-blames-economic-growth
[5]How Mindfulness can change your brain and improve your health, Harvard Medical School https://hms.harvard.edu/sites/default/files/assets/Harvard%20Now%20and%20Zen% 20Reading%20Materials.pdf
[6] National Health Interview Survey (NHIS) https://nccih.nih.gov/research/statistics/NHIS/2012/mind-body/meditation

1. A computerized reward system of digital assets for rewarding user behaviour beneficial to health characterised in that it comprises:
A monitoring unit including sensor means adapted to collect physical parameters from a user;
A validation unit comprising processor means programmed to correlate the physical parameters collected by the monitoring unit in order to detect the execution of a physical activity by the user;
A reward dispensing system, in communication with said validation unit, comprising a peer-to-peer network configured to manage a decentralized ledger blockchain distributed computing platform for inter-node communication; said reward dispensing system being programmed to generate and distribute peer-to-peer digital asset rewards if the validation unit detects a physical activity executed by the user.

2. Computerized reward system of digital assets according to claim 1, wherein the physical parameters are physiological, motion and/or body posture parameters.

3. Computerized reward system of digital assets according to any of the claims 1 and 2, wherein the monitoring unit comprises a body posture sensorial module and a respiratory effort sensor configured to detect the inhaling and exhaling breathing periods of the user.

4. Computerized reward system of digital assets according to claim 3, wherein the body posture sensorial module comprises at least on accelerometer, at least one gyroscope and at least one magnetometer.

5. Computerized reward system of digital assets according to any of the previous claims, wherein the monitoring unit further comprises a communication module programmed to establish a bidirectional wireless communication link with an external device; said external device being adapted to control the operation of the monitoring unit through said bidirectional wireless communication link.

6. Computerized reward system of digital assets according to claim 5, wherein the wireless communication link is performed over a wide area network, a local area network or a personal area network.

7. Computerized reward system of digital assets according to claim 6, wherein the communication link is Bluetooth.

8. Computerized reward system of digital assets according to any of the previous claims, wherein the monitoring unit is enclosed in a strap; said strap being wearable and adapted to be attachable to the chest or stomach of the user.

9. Computerized reward system of digital assets according to any of the claims 1 to 7, wherein the monitoring unit further comprises a meditation pad, suitable to perform meditation practices; said meditation pad including a proximity sensor, battery means, vibration means and an illuminated surface configured to emit visible light for relaxing purposes.

10. Computerized reward system of digital assets according to claims 3 and 9, wherein the visible light emitted by the pad's surface varies according to the parameters collected from the respiratory effort sensor.

11. Computerized reward system of digital assets according to any of the previous claims, wherein the processor means of the validation unit comprises:
a database structure adapted to store physical activity performance models, each of said models comprising a set of standard key-parameters that identifies unequivocally a certain physical activity; and
a correlation engine programmed to correlate the parameters collected by the monitoring unit according to special correlating schemes and to match the key-parameters resulting from a correlation scheme with the respective standard key-parameters of a physical activity performance model stored in the database structure.

12. Computerized reward system of digital assets according to claims 4 and 11, wherein the correlation engine is programmed to correlate any of the parameters collected from the respiratory effort sensor, from the set of at least one accelerometer, at least on gyroscope and/or at least one magnetometer.

13. Computerized reward system of digital assets according to any of the previous claims, further comprising a web user interface electronic platform, provided in a network-based system, said platform suitable to provide user access to reward system contents by means of a web application configured to communicate with said network-based system.

14. Computerized reward system of digital assets according to claim 13, wherein the reward system contents are related to activity session records, physical activity guiding sessions and reward data records.

15. Computerized reward system of digital assets according to any of the previous claims, wherein the digital asset is a cryptocurrency.

16. Computerized reward system of digital assets according to any of the previous claims, wherein the decentralized ledger blockchain distributed computing platform is Ethereum based.

17. Computerized reward system of digital assets according to claims 15 and 16, wherein the cryptocurrency is Ether.

18. Method for operating the computerized reward system of any of the claims 1 to 17, characterized by comprising the following steps:
Monitoring unit collects physical parameters from a specific user, generating a physical activity record;
Validation unit correlates the parameters of the physical activity record according to special correlation schemes, each correlation scheme comprising standard key-parameters defining a physical activity performance model, said model being stored in the database structure of the validation unit;
Validation unit performs a physical activity validation by matching the standard key-parameters of a special correlation scheme with key-parameters resulting from said special correlation scheme applied to the physical activity record;
If the physical activity record is valid, the physical activity record assigned to the specific user is uploaded to the reward dispensing system;
Reward dispensing system generates and distributes digital asset rewards to the specific user.

19. Method according to claim 18, wherein a physical activity is validated if the validation unit, for a special correlation scheme, detects a match between the key-parameters resulting from the correlation of the physical activity record and the standard key-parameters associated to the respective physical activity performance model.

20. Method according to any of the previous claims 18 and 19, wherein the amount of digital asset rewards that the specific user receives is a function between the total number of users with valid physical activity record and a daily number of digital assets that the reward dispensing system generates.

21. Method according to claim 18, wherein a physical activity is performed by the user during a physical activity session, said session comprising an initial stage and a final stage during which the monitoring unit collects physical parameters to generate a physical activity session record.

22. Method according to claim 21, wherein the validation unit performs a physical activity session validation, by correlating the parameters of the physical activity session record;

23. Method according to claim 22, wherein the physical activity session is validated if the validation unit, for a special correlation scheme, detects a match between the key-parameters resulting from the correlation of the physical activity session record and the standard key-parameters associated to the respective physical activity performance model.

24. Method according to claim 23, wherein if the physical activity session is valid the physical activity session record assigned to the specific user is uploaded to the reward dispensing system.

25. Method according to any of the previous claims 21 to 24, wherein the amount of digital asset rewards that the specific user receives is a function between the total number of users with valid physical activity sessions and a daily number of digital assets that the reward dispensing system generates.

26. Method according to any of the previous claims 18 to 25, wherein the digital asset rewards are distributed in periodic batches.

27. Method according to claim 26, wherein the digital asset rewards are distributed in daily batches.
